(19) United States
(12) Patent Application Publication
(10) Pub. No.: US 2018/0236189 A1
(43) Pub. Date: Aug. 23, 2018

(54) METERED DOSE INHALER AND SPACER WITH AIRFLOW AND HANDICAP ASSIST STRUCTURES FOR MAXIMIZING MEDICATION DELIVERY EFFECTIVENESS

(71) Applicants: Shawky Hassan, Grand Blanc, MI (US); Fikria E. Hassan, Grand Blanc, MI (US); Alexander Tarek Hassan, Ann Arbor, MI (US)

(72) Inventors: Shawky Hassan, Grand Blanc, MI (US); Fikria E. Hassan, Grand Blanc, MI (US); Alexander Tarek Hassan, Ann Arbor, MI (US)

(21) Appl. No.: 15/899,676

(22) Filed: Feb. 20, 2018

Related U.S. Application Data
(60) Provisional application No. 62/460,485, filed on Feb. 17, 2017.

(51) Int. Cl. A61M 15/00

(52) U.S. Cl. CPC A61M 15/0086; A61M 15/002; A61M 15/0018; A61M 15/0066; A61M 2205/3334; A61M 15/0091; A61M 15/009

(57) ABSTRACT

A metered dose inhaler having a body for receiving a medicinal canister. A lower mouthpiece end is in communication with an output valve of the canister for issuing an atomized medicinal spray. A plurality of apertures are defined along any of the sides or front and back walls of the body and above the output valve such that, upon depressing a trigger associated with the canister in combination with patient inhalation, airflows are generated which assist patient inhalation resulting in more efficient delivery of the spray and to better direct the spray into the patient's respiratory system. A further variant incorporates a motorized cap, such as for use by handicapped individuals who may be unable to actuate the metered dose inhaler due to anatomical or physiological disabilities.

### CROSS REFERENCE TO RELATED APPLICATIONS

The present application claims the priority of U.S. Ser. No. 62/460,485 filed Feb. 17, 2017, the contents of which are hereby incorporated by reference.

### BACKGROUND OF THE INVENTION

### Field of the Invention

The present invention is directed to metered dose inhaler (MDI) and spacer devices. More specifically, the present invention discloses a vented metered dose inhaler (Window-Haler) with an integral spacer design as part of the MDI structure or structurally independent spacer design that allows for a window for introducing air behind the actuated medication (Window-Spacer), all designs are constructed to maximize delivery efficiency of medication dosages by creating and directing an assist airflow, such as via patient intake/vacuum inducing air passageways however also contemplating a motorized battery powered mechanism for handicapped patients; this is triggered by the patient induced airflow when inhaling, or by the patient actuating any of temperature, infrared or touch, or Bluetooth sensors embedded in the passageways of the inhaled air by the patient for actuating the completed inhaler or spacer dose of medications into the patient's airways. The present invention acknowledges the difficulty of the prior art of MDI devices to synchronize patient inhalation with (push down) actuation of the inhaler, and the difficulty for patients with manual handicaps to push on the canister of medications to release the dose of medications. Such difficulties often result in markedly impaired efficiencies of medicinal delivery often as low as 15%.

Variants of the present design include configuration of the airflow holes (Windows) upon an outer housing/sleeve surrounding the MDI, such assisting in the commingling assist of an airflow behind the atomized dosage for oral delivery to the patient (not to be confused with motorized compressed air nebulizers). The versions also include a telescoping mouthpiece and also described is a motorized/power assist variant which can include both the compressed actuation of the MDI and/or airflow delivery assists as previously described.

### BACKGROUND OF THE INVENTION

The prior art is documented with numerous portable inhaler and related nebulizer devices, the purpose for which being the ability to orally administer an atomized medication to the airways and lungs of the patient, typically upon actuating a canister associated with the device in synchronization with the patient deeply inhaling efforts. Such inhalers provide main line treatment for patients who suffer from common obstructive and restrictive lung diseases (such as asthma and COPD).

An example of an existing MDI is shown at 1 in FIG. 5 (Prior Art) and includes, as best shown in the cutaway of FIG. 5, a canister 2 holding a reservoir of a medicament is provided and is contained within a plastic holder body, as further generally shown in cutaway at 3. A metering valve 4 is located at a lower end of the canister (such as shown being seated within an interior support location 4′ integrated into a lower interior position inside the canister and including a pressurized spring 5 and plunger 6 arrangement and which, upon being actuated via depressing motion (arrow 5) of the top of the canister 2 relative to the outer supporting body 3, downwardly displaced the canister 2 in a direction towards a lower internal support 7 configured within the inhaler interior. A passageway 8 is configured within the interior support 7 and, upon a lower atomizing inducing component 6′ in communication with the plunger 6 being caused to collectively displace in an opposite, inward and upward direction due to engagement with the support 7, causes a propellant (such as which can be charged within the canister) to be discharged through a metering valve integrating the lower atomizer 6′ associated with a lower end situated mouthpiece 9 integrally formed with the body 3 and to be delivered as an aerosol spray as depicted.

It is also noted that current MDI devices frequently fail to deliver the medications in the required dosages to the intended parts of the airways and lunges. In many studies, it has been estimated that only 15% of the inhaled medications reach their destination, with the other 85% escaping from the MDI to room air or is deposited over unintended tissues.

Other problems with existing MDI's include the unfulfilling design construction placing unreasonable demands on patient performance, this being exacerbated by the inability of the patient to synchronize their inhalation effort with the actuation of the medication canister in order to release the medications at the height of patient inspiration. With the lack of synchronization, the medications are only partially (or not at all) sucked into or driven to the respiratory tract. This problem is particularly acute in emergency (rescue) operations requiring immediate opening of the airways to prevent death by suffocation.

Other factors contributing to inefficient and/or improper MDI use include deposition of medications over organs other than where they are intended to go (tongue, gums, teeth, pharynx or larynx), deposition of medications on these other organs resulting in Dysphonia (harsh voice), cough, loss of voice and fungus infections on these organs, and deposition of medications on the mucus membrane of the trachea and large airways does invite fungus infection at these sites. Additional considerations include the patient maintaining a closed lips position to form a mouthpiece seal (see FIGS. 1-3) during dosage inhalation, such often resulting in total or partial resistance to medication flow given the creation of dead space in the patient's mouth.

Alternatively, maintaining lips in a loose seal position or spacing too far from the mouthpiece (Prior Art FIG. 4) can likewise result in inadequate delivery of the medication. Also known is the user of expander devices with the MDI, such constructed as spacers which attach to the mouthpiece of the MDI and which often contribute to the non-portability of the device owing to their bulkiness and awkwardness in use.

Kaar, U.S. Pat. No. 8,931,476, teaches an inhaler with an elongated aperture patterns formed in the housing. Of note, the air inlet apertures each extend in two different planes for assisting in creating a void space to prevent or limit covering of the opening from interrupting the airflow.

Other examples of known atomizers and MDI's in the prior art include Wilke U.S. Pat. No. 3,948,264, which teaches an inhalation device with an electrically driven vibratory mechanism for causing the medicament to be ejected from a capsule into a stream of inlet air.

The MDI of Nowacki, U.S. Pat. No. 4,534,343, teaches an upright cylindrical air chamber pre-pressurized with a medicament and a one-way valve and mouthpiece connected to a one-way diaphragm, with patient exhalation being bypassed to the outside.

Sladek, U.S. Pat. No. 6,039,042 teaches an MDI inhaler with an elongated inhalation mouthpiece/membrane and including both inhalation and exhalation valves.

Bruin, U.S. Pat. No. 9,427,534, teaches a drug delivery inhaler device incorporating an air flow rate indicator operable to indicate when the air flow rate along the air flow path is at or above a predetermined minimum level suitable for delivery of the drug to the patient.

Smith, USSN 2010/0000531 teaches a dry powder inhaler in which a torsional airflow intake is provided for assisting in medicinal delivery.

PCT/WO 95/07724 to Medtrac Technologies Inc. teaches a dry powder inhaler having electronic sensing and signaling which includes a monitor for prescribed dosages of medications received through a mouthpiece, an electronic housing for computing and recording when a proper amount of medicament is released associated with a proper amount of inhaled airflow and when the dispenser or inhaler is removed and replaced on the electronic housing. One embodiment of the invention includes an activation sheath received and secured to the dispenser. The electronic housing includes a first and second proximity reed switch for recording when a proper dose of medicament is released, and a fast response flow thermistor measures when sufficient airflow is being inhaled.

Finally, Stenzler USSN 2012/0247460 teaches an MDI spacer incorporating a one-way flow rate control valve and a collar for forming a seal about an aerosol nozzle.

### SUMMARY OF THE PRESENT INVENTION

The present invention discloses a metered dose inhaler having a body with an openable upper end for receiving a medicinal canister. The body includes a lower mouthpiece end in communication with an output valve of the canister for issuing an atomized medicinal spray.

In one variant, a plurality of apertures is defined at locations along the sides or the front and/or back of the body, and all are situated above (proximal) to the output valve such that, upon depressing a trigger associated with the canister in combination with patient inhalation, an airflow assisted patient inhalation is accomplished which resulting in more efficient delivery of the spray due to the surrounding directional assisting airflow generated by the passageways and in order to better direct the spray into the patient's respiratory system. In a further variant, the spacer if integrated in the structure of the mouth piece of the MDI, can be substituted by an optionally coiled tube structure instead of a straight tubing structure, thus resulting in a shorter less elongated and incorporated into the mouth delivery portion of the device, yet providing all the benefits of an ordinary straight commonly larger cylinder structure of the usual spacers.

A yet further variant incorporates a motorized cap, such as for use by handicapped individuals who may be unable to actuate the metered dose inhaler due to anatomical or physiological disabilities. The cap can be screwed or other affixed to a top inside location of the housing in fluidic communication with an interior of the medication canister supported within the main body, the cap houses a miniaturized electrical motor, battery and an airflow generating mechanism, with a sensor provided on the exterior of the cap (including without limitation infrared, thermal or Bluetooth sensor connectivity components) for activating the motor by the patient to influence a medicinal spray through the valve outlet and into the patient's mouth.

### BRIEF DESCRIPTION OF THE DRAWINGS

Reference will now be made to the attached drawings, when read in combination with the following detailed description, wherein like reference numerals refer to like parts throughout the several views, and in which:

FIGS. 1-4 present a series of environmental views of a variety of metered dose inhaler(s) (MDI) according to various embodiments of the present invention for inhaling medication associated with existing MDI designs, such including the prior (undesirable) technique of FIG. 4 for spacing the mouthpiece of the inhaler too far away from the user's lips;

FIG. 5 is a plan cutaway view of a metered dose inhaler according to the existing art;

FIG. 6 is a plan cutaway view of a modified metered dose inhaler according to one non-limiting variant of the present invention and which illustrates the pattern of side disposed apertures in the inhaler outer body for generating continuous and progressive airflow within the body interior in communication with the lower valve and spray outlet for increasing inhalation efficiency of the medication being issued;

FIGS. 7-8 depict a pair of illustrations of a further variant of the metered dose inhaler as shown in FIG. 6, and further depicting a plurality of telescoping sleeves for the mouthpiece which can be extended for use (FIG. 7) or collapsed (FIG. 8) during non-use;

FIG. 9 is a perspective illustration of the metered dosage inhaler of FIG. 6 again including side extending pluralities of airflow assist passageways for generating a continuous progressive airflow within the body interior, with potential additional apertures also position-able along any of side, front or back disposed surfaces for generating airway passages for mixing with the spray outlet for increasing inhalation efficiency of the medication;

FIG. 10 is an upper plan cutaway of a metered dose inhaler according to a further preferred embodiment and which includes a combination proximity sensor, electrical motor and portable battery integrated into a top securely attachable cap, with sensor-initiated actuation of the motorized cap drawing airflow through the apertures in the body and issuing a pressurized fluid output through a nipple or valve connecting the air flow inducing motor to an upper interior location of the medication canister installed into the MDI;

FIGS. 11-11A is an overall perspective of another variant of the spacer (also shown in environmental view in FIGS. 3 and 11A) which includes a reconfigured mouthpiece delivery portion of the spacer which is much less bulky because of its corrugated nature, provided by a plurality of corrugated loops extending within the interior of the main spacer body, and which provides almost the same inner surface area that the medication has to travel going towards the patient mouth, yet less bulky and smaller in size. This corrugated spacer design is structured as one corrugated tube-like independent or as an incorporated as an integral part of the structure of the inhaler outer sleeve, but can be used as an independent spacer device such that it provides the metered dose inhaler (MDI) with a built in but independent self-sufficient spacer equipped with the air window vented air stream via multiple holes (Windows) or sliding circular half B, over the other half A to create ventilation windows of different sizes in the wall of the spacer where the MDI gets inserted to delivery the medications and to match with the vital capacity of the patient; and

FIGS. 12-12B respectively illustrate each of a further perspective of the MDI device with the end opened for providing an airflow generation within interior of the device body (FIG. 12), a top view of the cover of the MDI end of the Window spacer (FIG. 12A) and a further end illustrating the space for inserting the MDI (FIG. 12B).

### DETAILED DESCRIPTION OF THE PREFERRED EMBODIMENTS

As will be described with reference to the several embodiments, the present invention discloses a metered dose inhaler which provides the ability to generate a continuous and progressive airflow within the body interior. This is accomplished in one variant via a series of side or front and back disposed airway passages, such air streams being drawn in through these windows by the patient inhalation effort, and will mix with and propel forward, the distally positioned actuated dose of medication, (the actuated dose of medication being closer to the patient mouth than the ventilation windows). This will augment the patient inhalation efficiency and enhance the speed of travel of the medication towards the patient lungs. This augmented airflow will have an added and very much welcomed beneficial effect on patients, by reducing the demand on them to exert sometimes unattainable amount of effort to drive the medication to their lungs, especially in cases where the lungs vital capacity is compromised by obstructive and/or restrictive lung diseases. A further variant of the MDI is designed specifically for handicapped patients, has an add on motorized cap component which includes a sensor and a built-in power supply in contact with base end of the medication canister for pressurizing the medication reservoir for assisted delivery through the mouthpiece. In a yet further related variant, an elongated, may be telescoping, mouthpiece with a progressively getting smaller diameter as it approaches the mouth of the patient. This gradually tapering extended mouthpiece, (spacer like device), is of overall progressively smaller size diameter until it reaches the patient mouth, will provide an added distance for the released medication to travel before getting to the patient mouth. This added travel distance for the medications, will enhance the synchronization of patient inhalation effort with the release and travel of such medication to the patient mouth, thus loss of medication (inherent in prior art MDI devices) is avoided. Also, this progressively smaller mouth piece will enhance the travel speed of the released medications resulting in practically zero waste of medications before reaching the patient mouth.

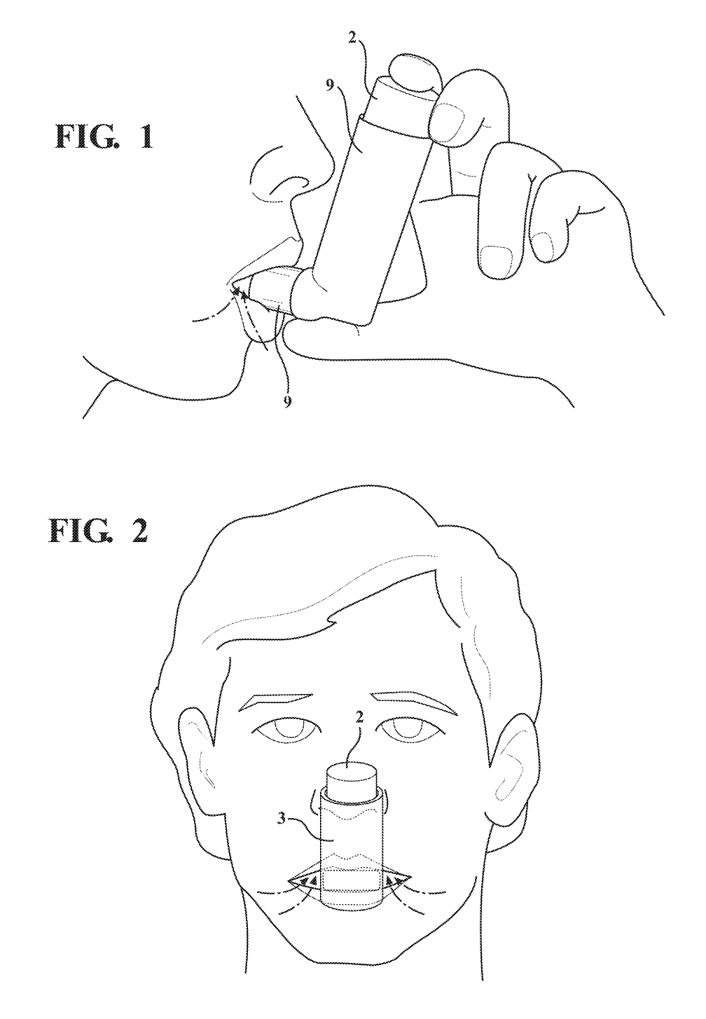

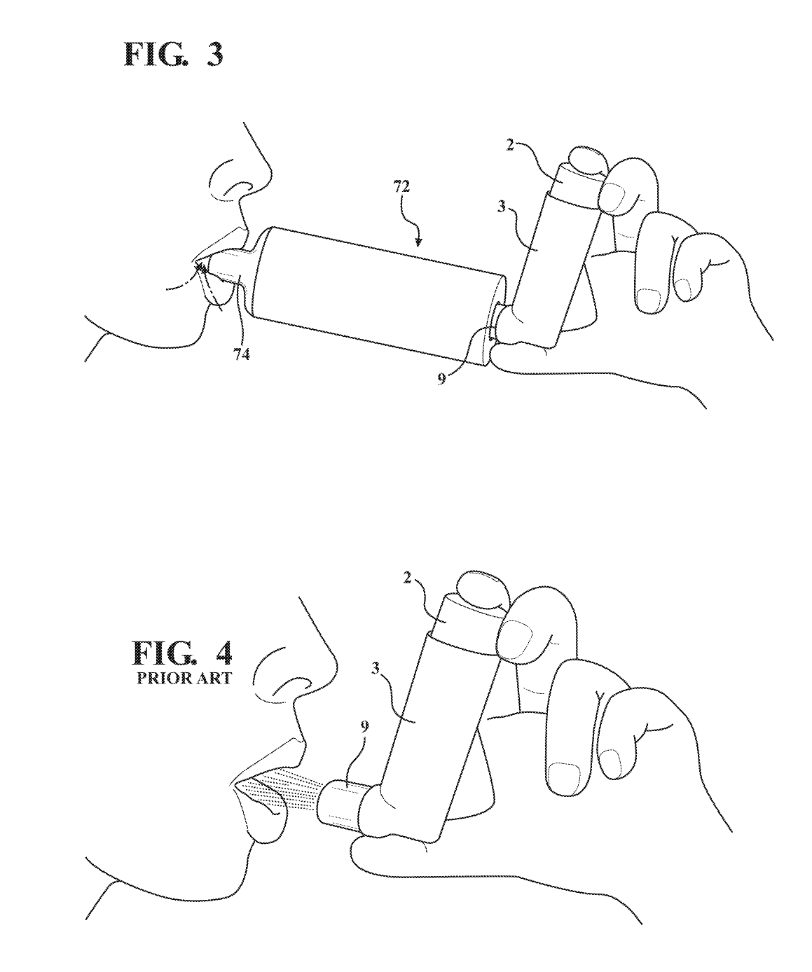

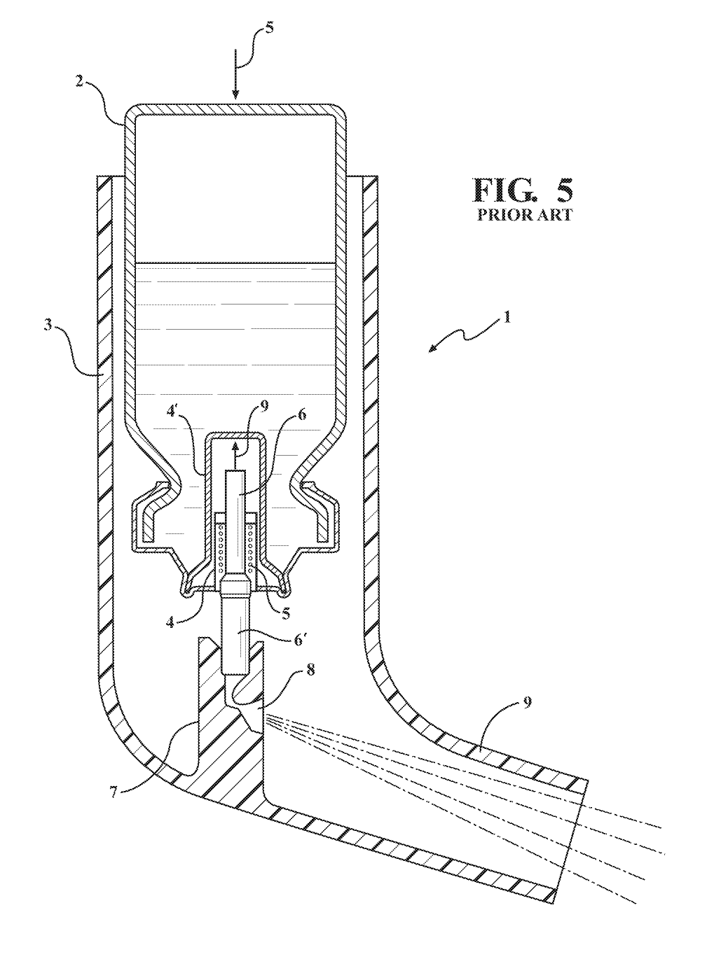

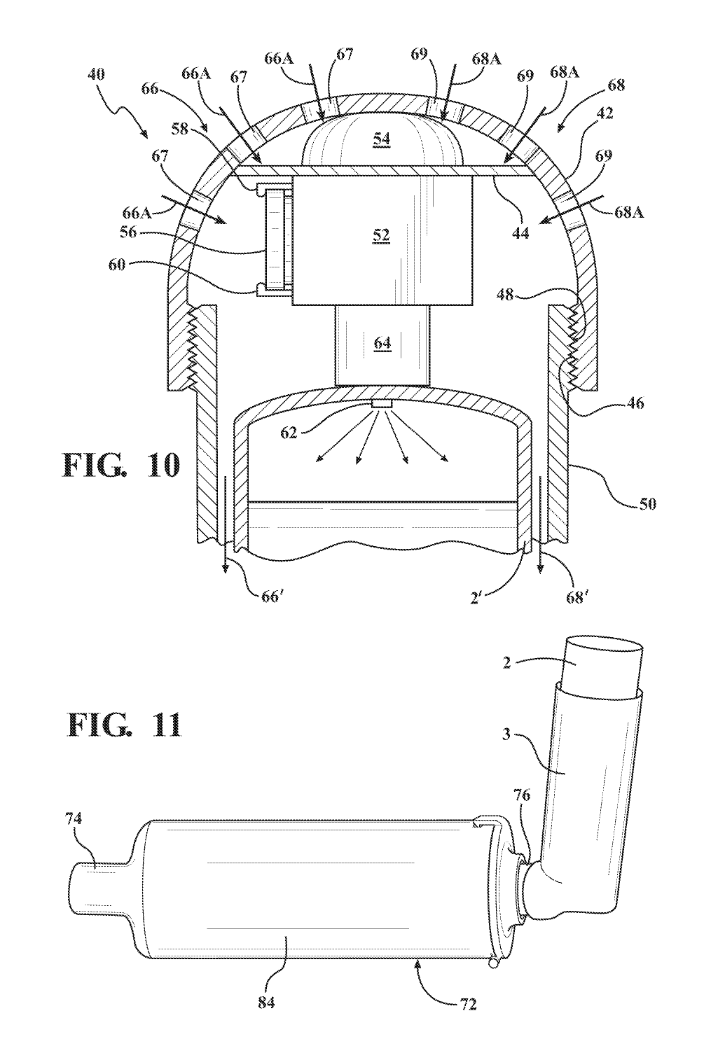

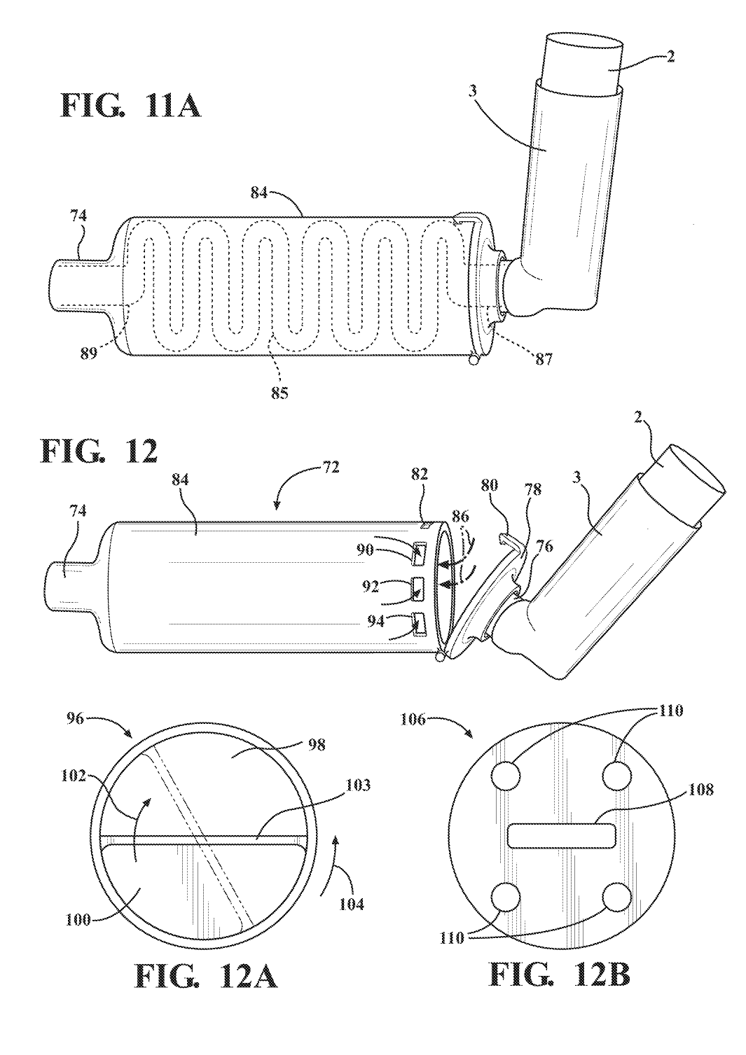

A description of a known type of metered dose inhaler is again referenced in FIG. 5 with the above-referenced description. FIGS. 1-4 illustrate a variety of operational views of metered dose inhalers, these including both the Prior Art variety of FIG. 5, as well as the variant of FIG. 6 (shown in FIG. 2), as well as that of FIGS. 11-12 (also depicted in FIG. 3). The purpose of the environmental views is to illustrate the correct technique for utilizing the MDI's for ensuring adequate delivery of the actuated medications. This includes a standardized delivery protocol as depicted in FIGS. 1-2 in which the mouthpiece of the MDI is placed between the user's lips and, upon the canister 2 being pressed downwardly, causing the atomized induced spray to be deposited directly into the user's mouth (oral cavity). FIGS. 1-2 further depict the technique of the airflows generated by the patient inhalation which pass between the mouthpiece and creviced sides of the patient's mouth into the oral cavity. While these airflows can, to some degree, be attendant in the application of the MDI devices according to any of the preferred embodiments, these are most attendant with the use of the Prior Art design of FIG. 5.

With reference now to FIG. 6, a plan cutaway view is depicted at 10 of a modified metered dosage inhaler according to one non-limiting variant of the present invention. The inhaler 10 largely replicates that shown at 1 in FIG. 5, with variations shown in the construction of the inhaler body, at 3′, and the mouthpiece, further at 9′. The device of FIG. 6 also includes patterns of side disposed apertures, see in phantom at 12, 14 and 16, configured into the inhaler outer body for generating a continuous and progressive airflow within the body interior in communication with the lower valve and spray outlet for increasing inhalation efficiency of the medication being issued.

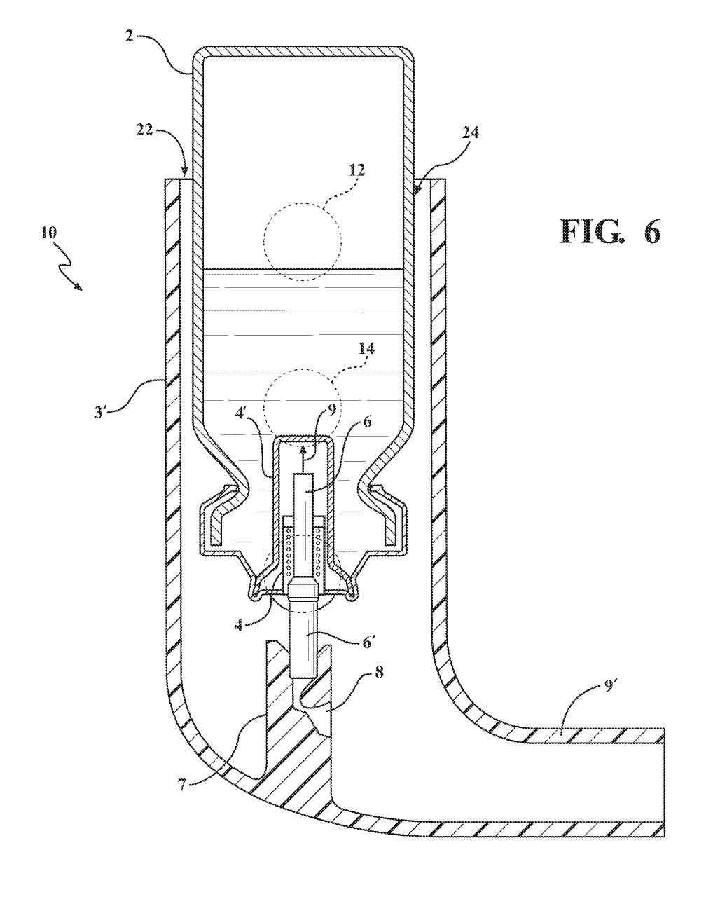

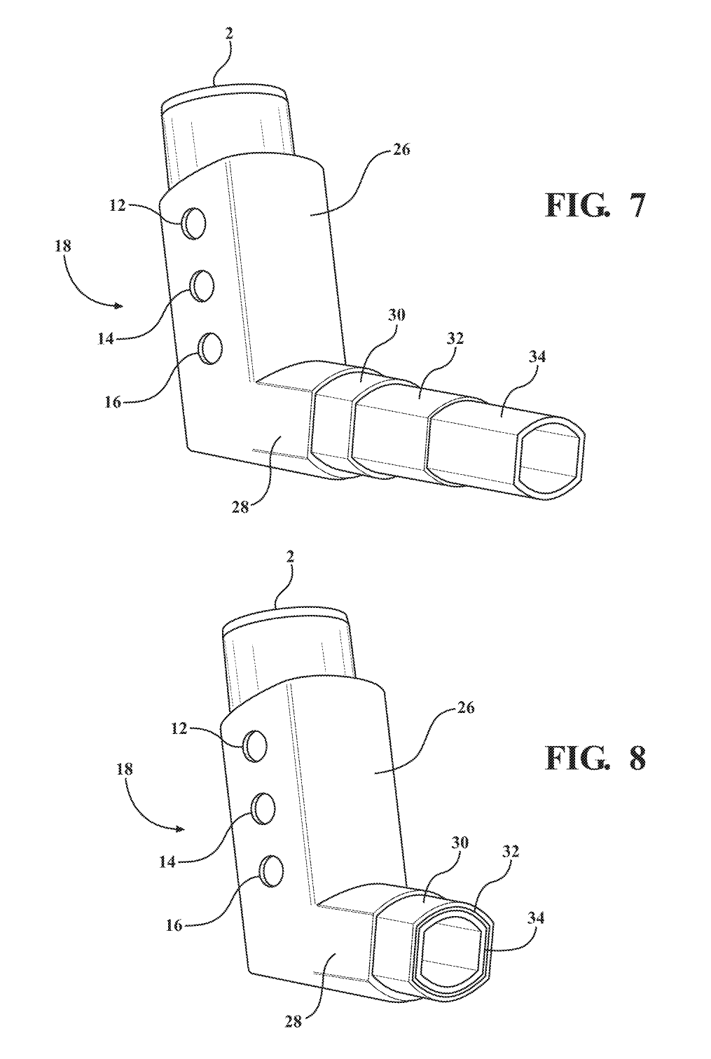

In each variant, the modified outer body (again at 10 FIG. 6, at 18 FIGS. 7-8, and further at 20 in FIG. 9) is provided for seating the MDI canister 2, within the body being configured the plurality of apertures or vent holes (again shown in each of these variants at 12, 14 and 16), and situated either on front/back or side walls of the MDI outer sleeve. As further shown, the aperture pattern is arranged in linear spaced fashion along the exterior of the body. Although not shown, it is understood that a matching plurality of vents or apertures can be likewise situated along a hidden or reverse/opposite side of the body. It is also understood (although not adequate, compared to the side or front windows) that the top of any of the inhaler bodies shown can include an expanded dimensioned or open space surrounding the upper end of the canister 2 (see at 22 and 24 in FIG. 6). This space did not prove to be adequate in the present-day MDI designs because of their smaller total space dimension, the location relative to the released medication from the canister, and the unavoidable chance of creating a back draft through which medications escape before getting to patients mouth.

The multiple air currents (also termed propeller air) enters from the ventilation windows into the space between the outer wall of the medication canister 2 and inner annular sleeve surface of the body, upon the patient initiating a voluntary inspiration effort. The number and arrangement of the windows or apertures can be modified in terms of shape, dimension and spacing and in order to generate air currents at a location above the metering or release valve which in turn create an effective driving force initiating behind and in a direction toward the outlet flow of the medication.

In this fashion, the induced airflow patterns provide additional driving force originating from behind and surrounding the medication for influencing the same at higher velocity and without any chance of back draft formations by which a large amount of inhaled medications escape the outlet flow and do not reach the lungs (see Prior Art explanation). The arrangement of the vents compensates for the lack of a free airflow behind the medication which is symptomatic of prior art MDI devices, as well as the lacking in synchronization between the triggering of the inhaler and patient inhalation and which, apart from decrease in medication delivery efficiency, further again causes the downside effect of incomplete medication delivery into the respiratory tract/system with resulting waste of expensive medications. The combination of the above features results in optimizing of MDI medication benefits by delivering more medication to the lungs without waste (into the surrounding air) or on other organs of the body and in particular during management of pulmonary obstructive diseases.

FIGS. 7-8 again depict a pair of illustrations of further variant 18 of the metered dosage inhaler, similar as shown in FIG. 6, and further depicting a reconfigured and more rectangular three dimensional shaped body, at 26, with an open top for receiving the canister 2. The lower end of the body further includes a reconfigured mouthpiece including a base integrated location 28, to which is telescopically mounted any plurality of individual and telescoping sleeves, these shown in one non-limiting variant at 30, 32 and 34 which are mounted to an inside perimeter of the base portion 28 for the mouthpiece and which can be extended for use (FIG. 7) or collapsed (FIG. 8) during non-use.

FIG. 9 is a perspective illustration, again at 20, of the metered dosage inhaler similar in construction to that depicted at 10 in FIG. 6, again including side extending pluralities of airflow assist passageways, previously shown at 12, 14 and 16 for generating a continuous and progressive airflow within the body interior, with potential additional apertures, see further in phantom at 12′, 14′ and 16′, also position-able along any of side, front or back disposed surfaces for generating airway passages for mixing with the spray outlet for increasing inhalation efficiency of the medication. Without limitation, the individual airflows induced through the aperture sides of the inhaler body 36 are not adequate as additional flows, as previously depicted at 22 and 24 in FIG. 6, induced through the gap in the open top of the device body (which is configured as further shown at 36 and terminating in a narrowed and rectangular shaped mouthpiece orifice as again depicted at 9′). Reference is made again, as mentioned before under [0033] to the limitations, untoward back draft effects and lack of effectiveness of the aperture sides of the inhaler body 36.

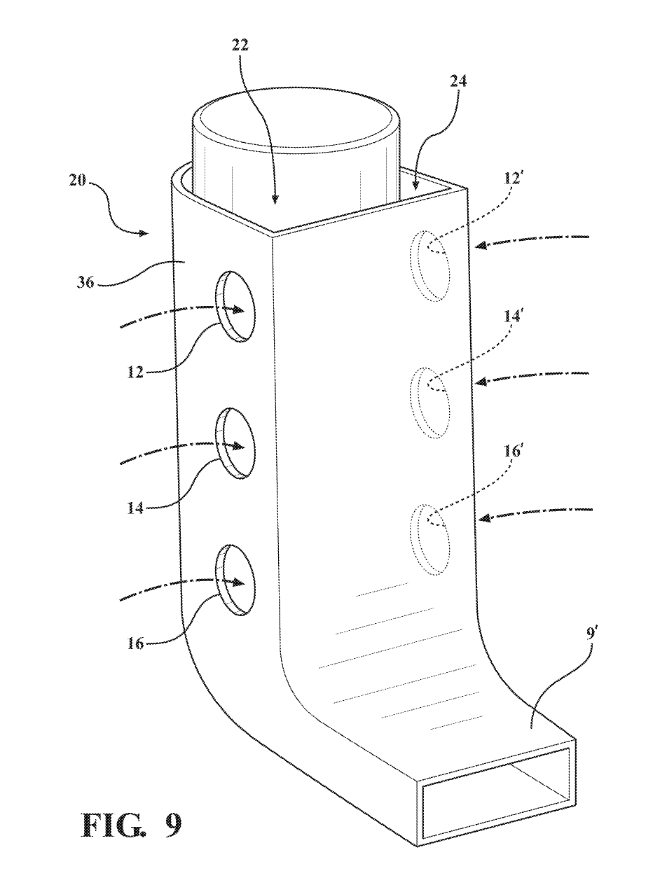

FIG. 10 is an upper plan cutaway of a metered dosage inhaler, generally at 40, according to a further preferred embodiment and which includes a dome (or other shaped) cap 42 having an interior support 44 for mounting the sensor, motor and battery. An inside lower perimeter edge of the cap 42 is configured with threads 46, these mating with opposing threads 48 configured within an uppermost and outwardly facing location of a main body 50 of the inhaler such that the cap can be securely screwed onto the open top of the inhaler body 50 following pre-installation of the medicament canister, this further shown at 2′ according to a reconfiguration as will be described below. It is further understood that the cap 42 can be configured according to any other shape additional to that shown and further that the threaded engagement profile shown can be replaced by any type of hinged, twist lock, tab and slot or other inter-engagement scheme for hingedly or removably attaching the cap to the open top of the inhaler body.

As further shown, the interior of the cap 42 includes, in combination, a miniaturized compressor style electric motor 52 of known construction which is mounted to an underside of the interior support 44 of the cap. Also included are a proximity sensor 54 mounted atop the electrical motor in proximity to the interior underside of the cap 42, along with a portable battery (such as a Lithium ion battery 56) mounted between receiving tabs 58/60 integrated into the housing of the motor and which communicates the battery to the motor contacts. A switch, or trigger, is integrated between the sensor 54 and battery 56 within the housing shown and, upon the sensor being activated in a manner to be described below, activates the electric compressor style miniaturized motor to cycle for a determined time interval in order to pressurize the interior of the canister.

As further shown, a nipple 62 projects from a fluid generating outlet 64 of the cap 42 which is in communication with the compressor style motor 52, the nipple communicating through the upper end of the modified medicament canister 2′. In operation, and upon the sensor 54 being activated (according to any of the operational protocols described below), the motor 52 is activated and draws in airflow, as shown at 66 and 68, from the several apertures (or windows) situated at the outer walls of the dome of the cap 42 (see further at 67, 69, et. seq.) above the base of the canister 2′. The airflow patterns can originate from the side window apertures in the cap 42 near its top, such being further directed downwardly between the inner wall of the main inhaler body 50 and the outer wall of the canister 2′ (see further at 66′ and 68′)

The motorized cap variant of FIG. 10 is particularly useful for handicapped individuals who are unable to actuate the MDI due to an anatomical or physiological disability of one or both hands. For such individuals, depressing of the canister to release the medications for the patient to inhale in the manner previously described and by pushing the base of the canister (not shown) against the inside lower support such as depicted at 7 in the prior variant of FIG. 6, can prove to be problematic.

The motorized cap variant 40 is to assist individuals with a handicap which makes it difficult for them to push the medication canister down to release the medication to be inhaled, and by triggering the motor to cycle for a given duration in order to generate a sufficient internal pressure within the canister reservoir in order to issue a discrete spray of medications through the orifice outlet (not shown) as an alternative to the operational protocol of FIG. 6. Without limitation, the sensor 54 integrated into the cap 42 can incorporate any of thermal or infrared triggering protocols. In another variant, the sensor can include a capacitive touch or other proximity trigger for activating upon the user placing the hand over the top of the cap. Alternatively, the sensor can be tied into any type of Bluetooth®, Near Field Communication, wireless or other proximity triggering protocol, such as which can be remotely triggered from such as a mobile phone utilizing a mobile application in communication with the sensor for issuing the medicament spray in the instance of complete loss of physiological hand function.

Proceeding to FIGS. 11-12, an overall perspective is shown of another variant of the externally attachable spacer device that can be attached to the metered dose inhaler (also shown in environmental view in FIG. 3), in which the inhaler 3 defines a first body and a separately attachable spacer, defined as a second body 72, which includes a reconfigured mouthpiece delivery portion 74 associated with the installed spacer. The mouthpiece 72 includes a spacer interior and extending portion which is elongated and which can be structured as one or more coiled tubes (a rearmost portion of which is depicted at 76 projecting from the back of the spacer) and incorporated as an integral part of the structure of the inhaler outer sleeve, such that it provides the metered dose inhaler (MDI) with an independent self-sufficient spacer. FIG. 12 is a further perspective of the spacer device with a hinged outer end cap 78 opened (via extending latch and end tab 80 and receiving seating aperture window 82 in the main inhaler body) for assisting in airflow generation within the interior of the device body.

The insertion of a spacer extension has a main body 84, the space between the medication release point (attached traditional mouthpiece 9 of the conventional inhaler body 3) from the canister again being depicted at 2 supported within a generic inhaler body 3, and such in turn being secured at its mouthpiece end 9 to the rear projecting end 76 of the coiled or extending portion configured within a main spacer outer body 84. FIG. 11A is a cutaway of the combination spacer and MDI of FIG. 11 and further illustrating a continuous interior conduit passageway 85 formed as a plurality of loops in a corrugated-like manner and extending within the main spacer body 85 between the MDI attaching end 87 and forward mouthpiece end 89. As previously discussed, the corrugated and multi-looped nature of the conduit 85 an approximate inner surface area that the medication has to travel going towards the patient's mouth, yet is less bulky and smaller in size. The conduit design can further be structured as any one or more tubes which can be independent or inter-twined in a manner which provides the metered dose inhaler with a built in and independent self sufficient spacer. Upon the patient's mouth being placed in communication with the forward mouthpiece location 74 associated with the spacer, the spacer provides a reservoir functioning as an inertia producing component where the velocity or speed of travel of the released medicament is reduced, allowing for the patient physiologic timing and speed of normal inspiration to match up with the speed of medication travel. Also, and while a prior art inhaler is depicted in the spacer operational view of FIG. 3, it is further understood that a side aperture or otherwise reconfigured inhaler, such as depicted in FIG. 6, can also be substituted for that shown.

The spacer component 72 also acts as a reservoir in which the medications are stored for a very brief period of time (up to a few seconds) following issuance from the canister 2 and travel to the interior of the main body 84, and before finally being inhaled by the patient. Relevant medical analysis and observation by one of skill in the relevant art notes that these few seconds of drug storage markedly reduce the urge/need and confusion panic of the patient to exactly synchronize the actuation of the medications from the MDI with the patient inspiratory effort, thus increasing both the efficiency and targeted delivery of the medicament to the patient's air passageways.

While it is acknowledged that all available spacers suffer from lack of a source of air current, (propeller air), to drive and propel not only some, but all of the medications which is already dispersed in the body of the spacer before it deposits by gravity or otherwise, to the walls of the spacer, the spacer construction described and shown constitutes a very efficient method to deliver the medicine to the patient lungs. As further best shown in FIG. 12, the hinged cap 78 may be pulled down (opened) by the patient after actuation of the MDI. In this arrangement, a large propeller body of air is generated (see airflows 86) behind the medication released from actuating the inhaler, and upon the patient starting inhalation. In order to operate as depicted in FIG. 12, all that is required is that the patient to pull down on the MDI after actuating it to open that window for propelled air (again depicted by currents 86) to be admitted when the patient inhales. Another structural alteration to the MDI end of the spacer again include multiple apertures (or windows) which are integrated in that end of the spacer (these depicted at 90, 92, 94, et seq. and configured on either side of the spacer main body 84) allowing for a stream of air brought into the spacer body and, most importantly, that stream of air is proximal to the location of the first MDI body 3, hence after actuation the air stream will be behind, not in front, of the actuated medications. This provides a source of propelling air generated behind the actuated medications and without the need for the patient to open the cover of the MDI end of the spacer which entails more work and may be an added confusion to operating the MDI and Spacer.

FIG. 12A depicts a top view, generally at 96, of a variant of a cover (compare to as previously shown at 78 in FIG. 12) integrated into the MDI proximate end of the spacer, corresponding to the attachment end location for receiving the MDI (previously shown at 76). A pair of first and second sides A and B correspond respectively to a wide open side and a solid surface side. The first side is also generally depicted at 98 and can represent an open space, with the second side further depicted as any of a flap 100 (can also include overlapping individual portions or be a single flap 100 which covers a closed portion of the spacer body). The flap 100 which can be pivoted (at 102) about a middle hinged location 103 or, in an alternate variant, slidably rotated (at 104) about a seating perimeter rim to create or adjust a dimension of of the open space associated with the associated MDI securing end of the spacer body 84. In this fashion, and upon pivoting or sliding the flap(s) 100 an overall window dimension represented at 98 is adjusted to match the specific patient vital capacity.

FIG. 12B further depicts, at 106, an alternate profile for the MDI receiving end and which includes a generally centrally located slot shaped aperture profile 108 for receiving the narrowed profile of the inhaler body (see at 9′ in FIGS. 6 and 9). Additional windows 110 of any plurality are also distributed across the surface area of the end cover to vary inhalation profiles and efficiencies to again match the specific patient vital capacity.

Beyond the feature of the spacer mouth delivery portion of FIGS. 11-12, as described herein, is understood and envisioned that any arrangement of an elongated structure can be provided in combination with any number or arrangement of integrated coiled tubes, and which provides the advantage of integrating a part of the structure of the outer sleeve of the inhaler, which effectively operates as an MDI with built in self-sufficient spacer. This negates the need for an added bulky extra device, namely an external spacer, that makes the MDI bulky and awkward to use. Furthermore, the mouth delivery portion of the MDI, is therefore elongated and coiled in the space between the medication release point from the canister and the patient mouth.

Additionally, and although the coiled and elongated mouth piece portion has a smaller volume compared to a regular size spacer, it will still function as an inertia introducing compartment where the travel speed of the released medications is reduced, to match the speed of the patient timing and speed of normal inhalation effort. In contrast, presently known spacers provide a fairly large reservoir for medications after their release from the canister, in which the medications are suspended before finally inhaled by the patient. Concurrently, drug suspension in a large volume compartment under the positive pressure initiated by the patient inspiratory effort to inhale the drug, enhances settling of the medication particles to the bottom of the spacers fairly large compartment.

In contrast to previous spacer devices, the present invention provides an elongated mouth piece of the MDI of relatively smaller volume to match the inhalation power and tidal volume of the patient, thus no loss of medication happens, as is the case in the large compartment of Prior Art spacers. That said, the spacer design of FIGS. 11-12 still provides for travel time of medications to help synchronize the patient inspiratory effort and actuation of the medications from the MDI.

Regardless of the embodiments disclosed (with partial exception of the motorized version of FIG. 10 the protocol for which is previously described), and consistent with the above description, one applicable medicinal delivery protocol for each of the manual inhaler variants, would include each of shaking the MDI, removing a cover off of the MDI mouthpiece (if applicable), extending the telescoping spacer portions (if applicable), forcing expiration of air from the lungs, placing the end of the spacer in the mouth and closing lips thereabout, inhaling deep with mouth closed tight, repeating after a predetermined time interval and, after use, covering the mouthpiece of the MDI for storage prior to reuse.

Having described my invention, other and additional preferred embodiments will become apparent to those skilled in the art to which it pertains, and without deviating from the scope of the appended claims. This can also include other modifications such as reconfiguring or relocating the vented air entranceway passageways from that shown, as well as constructing the MDI body from any of a plastic, acrylic or other stiff but thin material. The MDI upper sleeve portion of the body can also be constructed sufficiently wide (as well as sufficiently shortened) in order to accommodate most available sizes of canisters currently on the market. Also, retractable ridges will be situated protruding inwards from the inside wall of the MDI sleeve to support different size available canisters.

We claim:

1. A metered dose inhaler, comprising:
a body having an upper open end for receiving a medicinal canister, said body having a lower mouthpiece end in communication with an output valve of the canister for issuing an atomized medicinal spray;
at least one aperture defined in said body above the output valve; and
depressed triggering of the canister within the body, in combination with patient inhalation, resulting in more efficient delivery of the medicinal spray.

2. The inhaler as described in claim 1, said aperture further comprising a plurality apertures configured along at least one side of said body.

3. The inhaler as described in claim 1, further comprising at least one telescoping section extending from said lower mouthpiece end.

4. The inhaler as described in claim 1, further comprising said body being constructed of any of a plastic, acrylic or other stiff material.

5. The inhaler as described in claim 1, further comprising a spacer connected to the lower mouthpiece end of said body at a first end, said spacer having a second output end.

6. The inhaler as described in claim 5, the spacer further including at least one interior extending or winding coiled portion for reducing the total physical size of the spacer yet maintaining a delayed travel velocity, and increasing a time lag, between issuance of the medications from the canister and inhalation by the patient.

7. The inhaler as described in claim 5, further comprising a rear lid supporting said inhaler to said spacer, opening of said lid at a rear end of said spacer permitting airflow introduction into said spacer interior concurrent with patient inhalation, an additional mechanism being provided for permitting propeller airflow introduction into said spacer interior through a plurality of apertures in said first end of said spacer.

8. The inhaler as described in claim 7, further comprising a tab and seat configured between said lid and a rear location of said spacer to reclosing and securing said lid to the openable rear end of said spacer.

9. A power assist metered dose inhaler, comprising:
an inhaler body for receiving, through an open top, a canister with a medication holding reservoir, said body having a lower mouthpiece end in communication with an output valve of the canister for issuing an atomized medicinal spray;
a cap secured over said open top, an interior of said cap enclosing each of a miniaturized electric compressor motor, a portable battery power supply, and a sensor for triggering a timed cycling of the motor; and
a compressed fluidic generated output of said motor communicating with a top interior of the medications reservoir through a nipple extending between said motor and said canister interior in order to pressurize the interior of said canister during issuance of the medicinal spray.

10. The power assist inhaler of claim 9, said power supply further comprising a Lithium ion battery secured to a side of said miniaturized compressor electric motor in communication with contacts associated with a switch of said sensor and in order to trigger a timed cycling of said motor.

11. The power assist inhaler of claim 9, said cap having an interior structural support for retaining said motor, sensor and power supplying battery.

12. The power assist inhaler of claim 9, said cap having a dome shaped top with several apertures situated above said motor to propel air behind the actuated medications.

13. The power assist inhaler of claim 9, further comprising inter-engaging pluralities of threads configured between opposing rim edges of said cap and said open top of said inhaler body for permitting said cap to be screwed onto said body.

14. A combination metered dosage inhaler and spacer, comprising:
a first MDI body having an upper open end for receiving a medicinal canister, said first body having a lower end in communication with an output valve of the canister for issuing an atomized medicinal spray through an output valve;
a second spacer body connected to the lower mouthpiece end of said first body at a first end, said spacer body having a second mouthpiece output end; and
depressed triggering of the canister within the body, in combination with patient inhalation, creating an assisting directional airflow resulting in more efficient delivery of the medicinal spray into the patients respiratory system.

15. The combination inhaler and spacer of claim 14, said second body further including at least one interior extending or winding coiled portion for reducing a travel velocity, and increasing a time lag, between issuance of the medicament from the canister and inhalation by the patient.

16. The combination inhaler and spacer as described in claim 14, further comprising a rear lid supporting said first body to said second body, opening of said lid at a rear end of said second body permitting airflow introduction into said second body interior concurrent with patient inhalation.

17. The combination inhaler and spacer as described in claim 14, further comprising a plurality of apertures configured within said second spacer body proximate said lower end of said first MDI body permitting airflow introduction into said second body interior concurrent with patient inhalation.

18. The combination inhaler and spacer as described in claim 16, further comprising a tab and seat configured between said lid and a rear location of said second body for reclosably securing said lid to the openable rear end of said second body.

19. The combination inhaler and spacer as described in claim 14, further comprising at least one aperture defined in said first body above the output valve.

20. The combination inhaler and spacer as described in claim 19, said aperture further comprising a plurality of apertures extending along at least one side of said first body.

21. The combination inhaler and spacer as described in claim 14, at least one of said first and second bodies being constructed of any of a plastic, acrylic or other stiff material.
